(19) United States
(12) Patent Application Publication
(10) Pub. No.: US 2019/0326016 A1
(43) Pub. Date: Oct. 24, 2019

(54) Method of Capturing and Evaluation Uncertainty in Computerized Intelligent Systems for Medical Diagnosis

(71) Applicants: Thomas Andrew Kent, Houston, TX (US); Pitchaiah Mandava, Houston, TX (US)

(72) Inventors: Thomas Andrew Kent, Houston, TX (US); Pitchaiah Mandava, Houston, TX (US)

(21) Appl. No.: 15/956,432

(22) Filed: Apr. 18, 2018

(51) Int. Cl. G16H 50/20; G16H 10/20; G16H 10/60

(52) U.S. Cl. CPC G16H 50/20; G16H 15/00; G16H 10/60; G16H 10/20

(57) ABSTRACT

A method of collecting data as well as the uncertainty of that data for use in a computation intelligent system for use in distinguishing between a plurality of outcomes and discounting the probability of each outcome through a Bayesian theorem application of uncertainty in contributory elements for each outcome.

### CROSS-REFERENCE TO RELATED APPLICATIONS

This application claims priority under 35 U.S.C. § 119(e) from co-pending U.S. Provisional Patent Application No. 62/486,854, by Thomas A. Kent and Pitchaiah Mandava, “Incorporating Uncertainty of Multi-Streamed Information from Patient, Physician, Diagnostic Team into Computational Diagnosis and Treatment” filed 18 Apr. 2017, which, by this statement, is incorporated herein by reference for all purposes.

### STATEMENT REGARDING FEDERALLY SPONSORED RESEARCH OR DEVELOPMENT

The application does not contain any innovations made under federally sponsored research and development.

### BACKGROUND OF THE INVENTION

The field of medicine is increasingly utilizing computational systems to support diagnosis and treatment or other forms of intelligent informational analysis for patient care. Authors R. I. John and P. R. Innocent discussed in their paper “Modeling Uncertainty in Clinical Diagnosis Using Fuzzy Logic,” the use of fuzzy information on symptom intensity and duration in generating indexing in support of or to negate diagnosis of particular diseases.

During this time, they identified another problem which this innovation addresses. It was stated that a holistic approach to patient care is important. The authors discussed that the clinical reality of nursing requires nurses to make decisions arising from an ongoing holistic assessment of the patient need for nursing care, based on an extensive range of knowledge. Holistic assessment takes a number of environmental domains of patient need into account, as well as the need for clinical intervention.

Described herein is an approach to Computational Intelligent Systems (CIS) which incorporates information into an analysis and diagnosis which is believed to be more robust and accurate due to the consideration of data previously unutilized due to a lack of understanding of the data's contribution to the process of diagnosis as a whole.

CIS is seeing an increase in utilization as their capabilities improve. But it is difficult to depend on such systems knowing they consider all matters in a binary manner with little understanding of the human factors that go into a diagnosis and treatment of patients, which may be missing from the hard data inputted into the systems' decision engines.

As discussed here, CIS refers to soft computing techniques which are adaptable to multiple situations as contrasted with Artificial Intelligence (AI), which is based on hard computing techniques with a defined solution set. Soft computing includes, but is not limited to, neural networks, crisp systems, and fuzzy systems.

Crisp systems, to highly simply, consist of including an element in a set or not, and defining those sets so they lead to answers to particular questions. A similarly simplistic explanation of fuzzy systems is that they enable elements to be partially in a set according to a degree of membership (from 0 to 1) and not exclusively one of the two values. Neural networks take a set up input and produce output through a set of complex functions. The programming and operation of these types of systems are not a part of what is described here, and one skilled in the arts would appreciate many implementations of, and training methods for each of the types of soft computing systems, each of which can utilize the information presented here to gain improvement of the system's overall responses.

### BRIEF DESCRIPTION OF THE DRAWINGS

FIG. 1 illustrates a flowchart of a typical patient interaction through diagnosis in accordance with an exemplary embodiment of the invention.

FIG. 2 illustrates a patient input screen for capturing data in accordance with an exemplary embodiment of the invention.

FIG. 3 shows an exemplary custom input object for use in capture of patient data in accordance with an exemplary embodiment of the invention.

FIG. 4 shows an exemplary custom input object for use in capture of patient data in accordance with an exemplary embodiment of the invention.

### DETAILED DESCRIPTION OF THE PREFERRED EMBODIMENTS

More often than not in today's medical office, a History and Physical Examination (H&P Exam) is incomplete with patients unable to recall details from their past. Or the H&P does not get read by busy clinicians with heavy workloads. This results in valuable data being missed in the diagnosis and treatment process.

Additionally, patients are often too busy or distracted to complete lengthy website questionnaires. They may arrive for an appointment distracted or lacking focus, only to be presented with pages of forms to complete while in the waiting room, relying on their memory for pertinent details that may be key in determining a complete and proper diagnosis.

Patients in good health may have difficulty recalling the multitudes of medical history that accompany most adults today. Patients with current pain or discomfort may have trouble focusing or remembering details; or may simply omit details they feel are irrelevant or unimportant.

Those details may be exactly what a trained clinician finds necessary for determining a complete and proper diagnosis. Additionally, patients attempting to be fully compliant can still find it difficult to understand what is asked of them. They may be unable to formulate a response in a manner that is fully understandable to the clinical staff.

What is presented here are methods of standardizing and categorizing patient symptoms; capturing patient input in a reliable fashion; and including Levels of Reliability and Completeness (LRC) that express a confidence in the accuracy and thoroughness of the data presented or the data generated by the medical community in a diagnosis and treatment situation.

Before data's levels of reliability and completeness can be determined, it is important that the system itself does not introduce issues of inefficiency into the captured data. Therefore, quantitative and qualitative methods of capturing data in useable format may include using computers, tablets, digital ink, or microphones and natural language processors, or other computing devices.

Data may be standardized and categorized by organization, and query methods and/or the use of special components of graphical user interfaces (GUIs), or input techniques which guide a user and limit choices of language or description without truly restricting the data collected. Guiding a user's input may focus the user on specific issues that aid diagnosis, such as type, location, and intensity of pain.

For instance, if a patient's initial complaint is abdominal pain, an interface may present a graphical way to identify the level of pain in each of four quadrants of the abdomen. This data can be utilized to distinguish possible diagnosis.

As examples: right lower quadrant pain (Appendicitis, Meckel's diverticulitis, etc.) distinguished from left upper quadrant pain (splenic abscess/rupture, Gastritis), and multiple quadrants with similar levels, right and left lower quadrant pain (incarcerated hernia, torsion of ovarian cyst or teste, renal stone) vs. right and left upper quadrant pain (lower lobe pneumonia, acute pancreatitis).

One skilled in the art would understand that other diagnostic distinguishing input could be included to allow further distinction of the diseases presented above. Or the data may be coupled with other inputs from different areas of an H & P exam to narrow a diagnosis or establish probabilities for a diagnosis.

Sliders can indicate level of pain on a scale (1-10), or frequency (seldom-constant). Other interfaces can indicate other characteristics tri-slide may allow someone to position a multi direction marker among scales to indicate a unique type of paid between stabbing, burning, and tingling sensations.

One skilled in the art would appreciate that standard components of GUI and be utilized in unique manners by changing the tags and having different purposes for the component's values. In one embodiment, a series of radio buttons may be utilized to discuss a laceration, (i.e. jagged, weeping, scabbed, red, swollen, etc.) In another embodiment a color wheel may be utilized to determine discharge color or swelling and infection of a site.

Patients may assess their own knowledge and recall of information to apply an LRC value with various components of the data, or of a set of data. As an example, a patient may have a list of medications they brought from home and are completely positive of the medications, dosages, etc. which they have read directly off the bottle during data entry giving a higher level of reliability and completeness.

The patient may be less positive of their memory regarding their past surgeries and their family history (i.e. can't remember timing . . . “was the gallbladder surgery around Thanksgiving or around Easter of that year? I remember the kids were over for a family gathering.”) thus giving a lower LRC value. Doing so allows data with higher LRC values to be considered more influentially than data that is unreliable, or incomplete. Additionally, it allows incomplete data (lower LRC) to be completely or partially discounted in a diagnosis.

The same methods apply to the work of clinicians. While we will assume reliability in the clinician's own findings, the H&P Examination allows opportunity for the Clinician to further assess the LRC of patient data such as the confidence they have in stating symptom onset as well as medicines, allergies etc. It may be useful LRC data to determine a patient's alertness, focus, and cognizance.

Finally, Diagnosticians have always had a reliability issue. Imaging may have a bad angle, contrast may make distinguishing elements difficult. Tests may yield results that are “borderline normal” in one area and/or “borderline abnormal” in another. Some test types may trade conclusiveness for time and cost.

Additionally, interpretation of images is an art form that requires unique skill and can be influenced by many factors. There is always the diagnosticians' own confidence, as well as their “track-record” in general and in the specific type of interpretation being performed. These factors contribute to assigning an LRC value to interpretations and testing results.

By assigning LRC values to data collected a better diagnosis can be determined. Fuzzy logic systems, Machine Learning (ML), and Deep Learning (DL) algorithms may take additional input from hospital wide or patient registry databases consisting of established diagnosis of patients and patient histories to determine a list of possible diagnosis. LRC values may be used by a system to determine threshold values for inclusion or exclusion of certain data during a database query for possible diagnosis. LRC values that have been modified to reflect a plurality of LRC values may be utilized as an input to a database or may be a threshold for inclusion or exclusion of certain data elements from a database query.

Deep Learning is a natural culmination of Machine Learning. Deep Learning refers to layers of processing. These successive layers can be fuzzy models, neural network models, etc. Each layer successively progressing towards a better answer. A Deep Learning computation module can be a part of a data input interface and/or part of an aggregator, querying, or intelligent processing components.

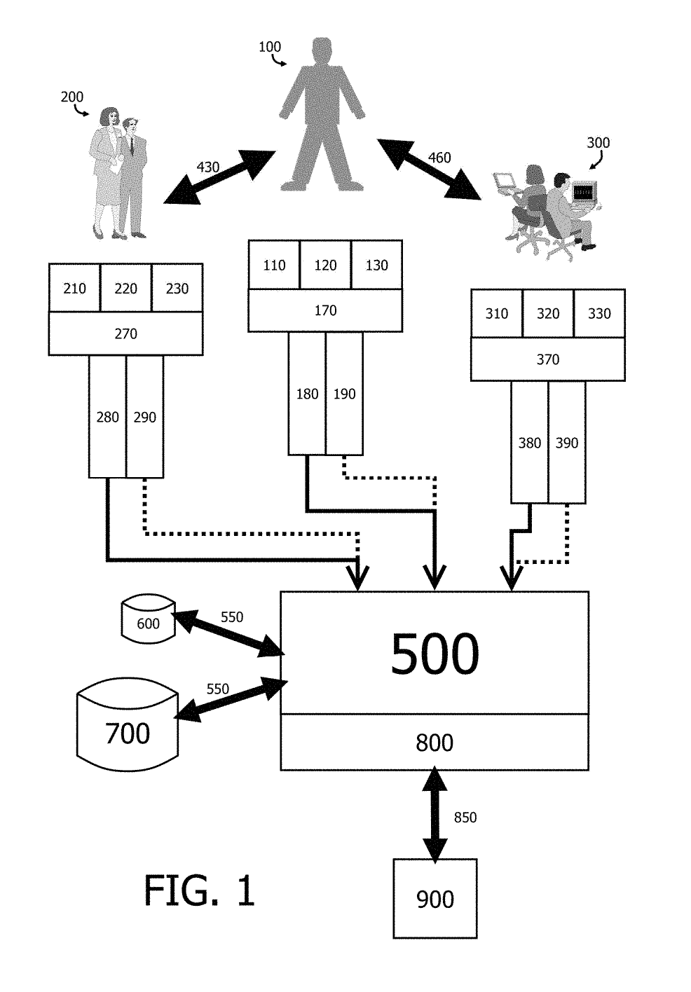

In the preferred embodiment, it is preferable to consolidate system intelligence into the intelligent processing components rather than spreading DLs to the data interfaces (represented as 170, 270, & 370 in FIG. 1). In the preferred embodiment, the intelligent processing components (represented as 500 and 800 in FIG. 1) contain DL, as a component of the aggregation functions for determining successive queries and which data may be included or omitted from those queries.

Additionally, the intelligent processing components contain DL in the report generators (represented as 800 in FIG. 1) as post processing, after querying the databases, to apply discounting of probabilities from the uncertainty associated with data utilized in the queries. One skilled in the art would appreciate that DL may be incorporated into the modules in numerous ways and various configuration, and that all have distinct advantages and disadvantages; the considerations of which are not important here as all are considered included in this innovation description.

An overall LRC value, (a modified LRC value) may be determined if similar data is collected with individual LRC values from a patient information output stream, a clinician information output stream, and/or a diagnostic information output stream. This value could be determined by an application of some mathematical formula. Principals applied may be based on, but are not limited to: Bayesian theory, weighted averages, averaging, or other methods of aggregating a plurality of values.

The data considered in each diagnosis may be coupled with the LRC values or the modified LRC values to apply a Bayesian adjustment to the probabilities resulting in a final report with a plurality of diagnoses and probabilities of likelihood for each.

The concept of uncertainty in medical decision making has been noted on occasion in the computerized learning literature and has been mentioned in a prior art (Zhang et al and John R. l. et al). However, in the process described here, uncertainty (along with completeness or lack thereof) is explicitly captured throughout the medical diagnosis process. This produces additional data rather than by specifying a specific analytical method, i.e. fuzzy logic.

The additional data has significant use in generating more reliable diagnosis and treatments. By capturing uncertainty through the interface, not just from the patient, but also with the patient by the clinician and with the patient by the diagnostician, as well as the by the diagnostician based on factors of his performance (i.e. test types, equipment quality, contrast issues, angles, patient compliance during testing, etc.), any computerized intelligence system currently developed or yet to be realized can be employed using a variety of uncertainty algorithms to influence the outcome of the computerized diagnosis. Multiple outcomes can be discounted by the uncertainty of data utilized in producing each outcome, or with multiple uncertainty assessments by various actors in the process.

Uncertainty was a by-product of the method of analysis in prior art. Herein there is a fundamental difference in that uncertainty, completeness, and reliability is a required component supplementing the data gathered and utilized to enhance results produced from the data with and without uncertainty being considered by the methods used to produce diagnosis. Uncertainty becomes data and may be utilized as an additional data during diagnosis.

### DETAILED DESCRIPTION OF THE DRAWINGS

FIG. 1 illustrates a flowchart of a typical patient interaction through diagnosis in accordance with an exemplary embodiment of the invention. A patient (100) presents with illness or injury. A History and Physical (H&P) report must be completed. This is often started with questionnaires to the patient for self-reporting.

Initially the patient may provide information via computing device input to provide a current illness/complaint (110) which may include their chief complaint, and the history of the present illness or injury. Histories (120) includes, but is not limited to: surgical history, medical history, social history, and family history. Other pertinent patient information (130) includes allergies, medications, a review of systems.

Each piece/collection/stream of data may be assigned a level of reliability and completeness. These can be assigned to distinct levels of resolution among the data point/categories/suppliers/sources.

These LRC values may be self-assessed; or may be, for example, determined by comparison with past-collected data, or may be measured by input characteristics, including but not limited to haptic technology, entry time, handwriting analysis, vitals, etc. One skilled in the arts would appreciate that there are various techniques of sensory perception, both electronic and biological which can provide a determination of the accuracy/confidence/trustworthiness (intentional or unintentional) of information received.

Information is collected through the patient data interface (170) which renders data into an electronic format for input into the computational information system. The patient data interface may include, but is not limited to: computers, websites, tablets, microphones coupled with natural language processors, digital ink, smart phones, and other smart computing devices.

Data from the patient data interface (170) produces a patient information output stream (180) and an LRC stream (190). The patient information output stream (180) may include, but is not limited to: complaints, descriptions, symptoms, histories, pain & discomfort levels. The LRC stream (190) may include but is not limited to: family history completeness and correctness; patient history completeness and correctness; present illness onset, symptom severity, symptom completeness.

Once patient self-reported information is collected the patient will see a clinician (200) for a physical exam (430). The clinicians (200) (doctors, nurses, specialist, etc.) will gather their own information in the form of a patient interview (210), observations (220), and interactive test (230) which include the physical part of the physical exam and results in measured vitals, palpatory exam results, etc.

This information is entered into the clinician data interface (270) which produces a clinician information output stream (280) and a clinician LRC stream (290). The clinician information output stream (280) includes, but is not limited to: complaint, focus, attention/alertness, vitals, tenderness, locations of pain, misalignments, observations, degrees of symptoms, suspected diagnosis, treatments of symptoms.

The LRC stream (290) may include but is not limited to: suspected degree of withholding; intentional or unintentional noncompliance; confidence levels of clinician; familiarity of clinician with suspected illness.

The clinician can order diagnostic testing (460). Diagnosticians (300) (radiologist, pathologist, lab technicians, x-ray technicians, imaging specialist, etc.) perform diagnostic testing (460) and generate results such as imaging and interpretations (310), lab test results (320), and other diagnostic test with results and/or interpretations (330).

Diagnostic testing results, images and lab test are often created by computing devices and are often in digital formats; or may be entered into the diagnostic data interface (370) which produces the diagnostic output stream (380) and the diagnostic LRC stream (390). The diagnostic information output stream (380) includes, but is not limited to: x-rays, MRIs, CT-scans, interpretations, radiologist reports, pathologist reports, etc.

Interpretations by diagnosticians is an art form that requires unique skill and can be influenced by many factors. The diagnostic LRC stream (390) attempts to account for many of these. The diagnostic LRC stream (390) may include but is not limited to: impressions of the technicians such as bad angle on imaging; contrast issues making elements difficult to distinguish; the diagnosticians' confidence; the diagnostician's “track record” in general and in the specific type of interpretation being performed. These factors contribute to assigning an LRC value to interpretations and testing results.

The data streams of traditional data (180, 280, 380) are supplemented with LRC steams (190, 290, 390) represented by dashed lines which all are input into the computation information system (500) which aggregates all data for input into algorithms which query (550) databases (600, 700) for determining diagnosis and treatment possibilities.

The LRC streams (190, 290, 390) may not be weighted equally in some embodiment. Trained clinicians may be better and more reliable at identifying uncertainty than that which a patient self-reports. Diagnosticians may be hesitant to report LRC issues which could be considered to reflect poorly on their own performance.

Local patient data/hospital data/regional data may be in databases (600). While other global databases (700) may include machine learning data sets, results, for use in training, as additional adaptation, or evolution of the computation information system (500).

After processing, the computation information system (500) generates a plurality of diagnosis with probabilities. Those are sent to a report generator (800) which may take each diagnosis, it's probability, and the inputs of patient data stream (180), clinician data stream (280), and diagnostician data stream (380) contributing to the diagnosis and apply Bayesian theorems to further adjust the probabilities before producing (850) a diagnostic report (900). One skilled in the art would appreciate that other mathematical formulas may be applied beyond the straight Bayesian described above. Such other mathematical formulas are intended to be included in the application of the innovation described herein, and not to limit to only a single theorem.

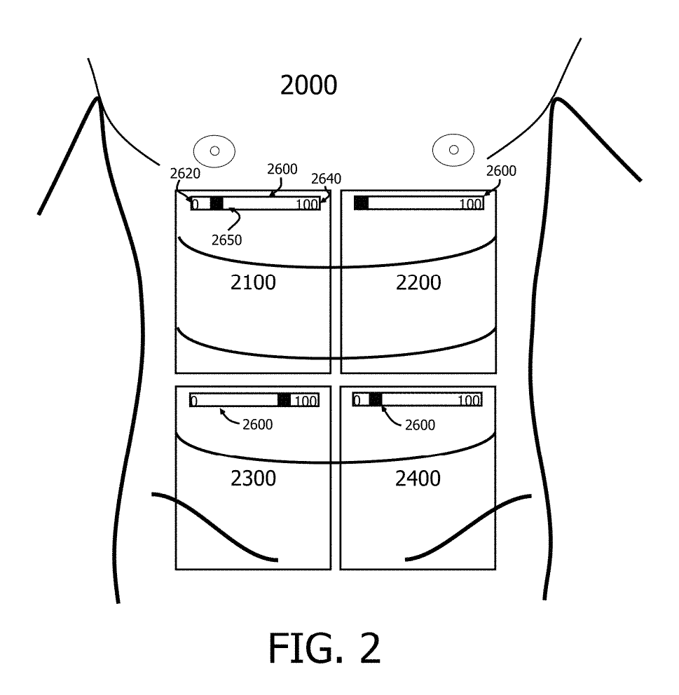

FIG. 2 illustrates a patient input screen for capturing data in accordance with an exemplary embodiment of the invention. In an exemplary scenario a patient's chief complaint may be abdominal pain. An adaptive patient interface, as a result, presents the interface (2000) for capturing patient input.

In the interface (2000), the abdomen illustration is divided into quadrants (2100, 2200, 2300, and 2400). Each quadrant includes an input scale (2600) for indicating the level of discomfort. Each input scale (2600) has a bottom of scale value (2620), here indicated as 0, and a top of scale value (2640), here indicated as 100. A slider (2650) is positioned to indicate the relative values.

As an example, the lower right quadrant's (2300) slider (2600) is positioned near the top of the scale, the upper right and lower left quadrant's (2100, 2400) sliders (2600) are only slightly elevated, and the upper left quadrant's (2200) slider (2600) is at the bottom of the scale. This reading could lead to a possible diagnosis of appendicitis. However, this is only one possibility, other input could make this a low possibility diagnosis.

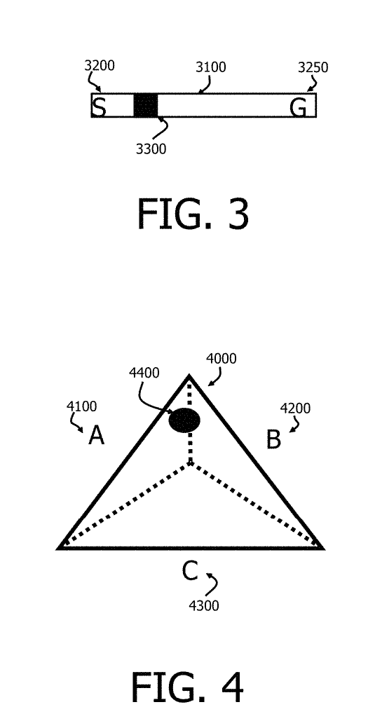

FIG. 3 shows an exemplary custom input object for use in capture of patient data in accordance with an exemplary embodiment of the invention. The input scale (3100) indicates a degree of difference between two values. The bottom of scale value (3200), indicated as an ‘S’, and the top of scale value (3250), indicated as a ‘G’ may be used to report the onset of a symptom in a continuous possible range from ‘sudden’ to ‘gradual.’

FIG. 4 shows an exemplary custom input object for use in capture of patient data in accordance with an exemplary embodiment of the invention. The tri-point input scale (4000) selects between three non-exclusive values in varying degrees.

As an example, this tri-point scale may indicate pain sensations and reports varying amounts on the A Scale (4100) indicating a “stabbing pain”, the B Scale (4200) indicating a “burning pain”, and the C Scale (4300) indicating a “tingling pain”. The A, B, C labels could be replaced with any value and may be translated into other languages. An input point (4400) is moved to indicate the composition of pain. Indicated is a “stabbing-burning” pain which has slightly more stabbing sensation.

One skilled in the art would appreciate that these are only a few examples of the input interfaces which may be used to categorize and clarify data from un-trained patients, as well as being used for limiting mistakes by clinicians, and diagnosticians.

The flow diagrams in accordance with exemplary embodiments of the present invention are provided as examples and should not be construed to limit other embodiments within the scope of the invention. For instance, the blocks should not be construed as steps that must proceed in a particular order.

Additional blocks/steps may be added, some blocks/steps removed, or the order of the blocks/steps altered and still be within the scope of the invention. Further, blocks within different figures can be added to or exchanged with other blocks in other figures.

Further yet, specific numerical data values (such as specific quantities, numbers, categories, etc.) or other specific information should be interpreted as illustrative for discussing exemplary embodiments. Such specific information is not provided to limit the invention.

In the various embodiments in accordance with the present invention, embodiments are implemented as a method, system, and/or apparatus. As one example, exemplary embodiments are implemented as one or more computer software programs to implement the methods described herein.

The software is implemented as one or more modules (also referred to as code subroutines, or “objects” in object-oriented programming). The location of the software will differ for the various alternative embodiments. The software programming code, for example, is accessed by a processor or processors of the computer or server from long-term storage media of some type, such as a CD-ROM drive or hard drive.

The software programming code is embodied or stored on any of a variety of known media for use with a data processing system or in any memory device such as semiconductor, magnetic and optical devices, including a disk, hard drive, CD-ROM, ROM, etc. The code is distributed on such media or is distributed to users from the memory or storage of one computer system over a network of some type to other computer systems for use by users of such other systems.

Alternatively, the programming code is embodied in the memory (such as memory of the handheld portable electronic device) and accessed by the processor using the bus. The techniques and methods for embodying software programming code in memory, on physical media, and/or distributing software code via networks are well known and will not be further discussed herein.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A method of rendering a medical diagnosis with a computational intelligent system comprising:
capturing current illness data from a patient;
capturing history data from the patient;
capturing additional pertinent patient informational data from the patient;
assessing reliability of data collected from the patient;
utilizing a subset of the data collected to query a database for a one or more possible diagnosis solutions matching the subset;
determining a probability of each solutions being the final solution;
discounting the probability in accordance with the reliability of data in the subset.

2. A method, as described in claim 1 wherein assessing reliability of data collected from a patient comprises asking the patient for a reliability level from a predefined scale.

3. A method, as described in claim 1 wherein assessing reliability of data collected from a patient comprises comparing data supplied by the patient to known data located from another source.

4. A method, as described in claim 1 wherein the subset of data collected comprises data associated with reliability levels above a threshold value.

5. A method, as described in claim 1 wherein discounting the probability in accordance with the reliability of data in the subset comprises applying a Bayesian theorem to each probability for one or more data in the subset having a reliability assessed.

6. A method, as described in claim 1 further comprising, before utilizing a subset of the data:
the patient receiving a physical exam from a clinician;
wherein the physical exam comprises, in addition to the physical examination:
an interview with the patient,
clinician observation of patient, and
clinician conducting interactive test on/with the patient;
capturing interview data from clinicians;
capturing observation data from clinicians;
capturing results of interactive test and a physical exam of the patient from clinicians;
assessing reliability of data collected from the clinicians.

7. A method, as described in claim 6 wherein assessing reliability of data collected from the clinicians, further comprises collecting a reliability of patient data determined by clinician's assessment of a patient's withholding or noncompliance.

8. A method, as described in claim 7 wherein assessing reliability of data collected from the clinicians, further comprises collecting a reliability of patient data determined by clinician's assessment of if a patient's withholding or noncompliance is intentional or unintentional.

9. A method, as described in claim 6 wherein assessing reliability of data collected from the clinicians, further comprises determining clinician's confidence level and/or familiarity with suspected illnesses.

10. A method, as described in claim 1 further comprising, before utilizing a subset of the data:
the patient receiving one or more diagnostic test from a diagnostician;
wherein the diagnostic test comprise one or more of:
x-ray, MRI, CT-Scan, EKG, specimen collection, and/or lab work wherein the diagnostic test further comprise one or more of the following:
reports, interpretation, readings, expert analysis, test results, images, and lab results (together referred to as diagnostic results);
capturing diagnostic results;
capturing reliability data of diagnostic test;
capturing reliability of diagnostician;
assessing reliability of data collected from diagnostic test;
assessing reliability of diagnosticians.

11. A method, as described in claim 10 wherein capturing reliability of data of diagnostic test comprises assessing one or more of the following:
contrast issues, bad angles of image, inferior equipment, equipment's need for calibration and/or alignment.

12. A method, as described in claim 10 wherein assessing reliability of diagnostician comprises:
determining diagnosticians confidence level in the specific test performed and the results and/or determinations made; and
assessing experience of diagnostician with the specific diagnostic test performed.

13. A method, as described in claim 1 wherein discounting the probability in accordance with the reliability of data in the subset comprises:
determining data of the subset utilized to query the database for one or more possible diagnosis solutions;
determining reliability of data collected;
discounting the probability of each solutions being the final solutions by applying Bayesian theorem to each probability for the reliability of each data utilized to query the possible diagnosis.

14. A method, as described in claim 13 further comprising presenting the one or more possible diagnosis solutions, along with the discounted probabilities associated with each possible diagnosis as a final determination of the system.

15. A method of rendering a medical diagnosis comprising:
having an application;
capturing current illness data from an application user;
capturing history data from the user;
capturing additional pertinent informational data from the user;
assessing reliability of data collected from the user;
utilizing a subset of the data collected to query a database for a one or more possible diagnosis solutions matching the subset;
determining a probability of each solutions being the final solution;
discounting the probability in accordance with the reliability of data in the subset.

16. A method, as described in claim 15 wherein assessing reliability of data collected from a user comprises asking the user for a reliability level from a predefined scale.

17. A method, as described in claim 15 wherein capturing current illness data from a user further comprises:
presenting the user with input screens focused on the current illness, and/or probable symptoms as determined by analysis of previously entered current illness data.

18. A method, as described in claim 17 wherein input screens focused on the current illness, and or probable symptoms are configured with inquiries which distinguish between one or more possible diagnosis solutions.

19. A method, as described in claim 15 wherein the application is a website.

20. A method, as described in claim 15 wherein the application:
saves user data to files;
loads user data from files; and
accepts edits of data from user prior to querying a database.
